(19) United States
(12) Patent Application Publication
(10) Pub. No.: US 2026/0308663 A1
(43) Pub. Date: Oct. 8, 2026

(54) ENZYMATIC CONVERSION OF METHYLATED NUCLEIC ACIDS FOR SEQUENCING

(71) Applicants: Roche Sequencing Solutions, Inc., Pleasanton, CA (US); Kapa Biosystems, Inc., Wilmington, MA (US)

(72) Inventors: Shwu shin Chang, Dublin, CA (US); Nathalia Graf Grachet, Hayward, CA (US); Daniel Klass, Lake Bluff, IL (US); Nicole Jacinda Lambert, Cambridge, MA (US); David Lawrence Penkler, San Ramon, CA (US); Peter Gustav Ristow, Cape Town (ZA)

(21) Appl. No.: 19/483,388

(22) PCT Filed: May 6, 2024
(86) PCT No.: PCT/EP2024/062405

§ 371 Date: Nov. 11, 2025

Related U.S. Application Data
(60) Provisional application No. 63/466,190, filed on May 12, 2023.

(51) Int. Cl. C12Q 1/6806; C12Q 1/6855

(52) U.S. Cl. CPC C12Q 1/6806; C12Q 1/6855

(57) ABSTRACT

The present disclosure relates, in general, to the enzymatic conversion of methylated nucleic acids in order to distinguish between methylated and unmethylated cytosines in DNA and, more particularly, to improved methods and compositions for enzymatic methylation sequencing. In one aspect, diverse compositions and methods are provided for improved recovery of methylation signal. The methods include, one or more of: a nick repair step, restoration of methylation signal, use of modified methylcytosine nucleic acid adaptors, and use of helicase, ssDNA binding proteins, engineered DNA ligase, or a combination thereof.

### CROSS-REFERENCE TO RELATED APPLICATIONS

The present disclosure claims the benefit of the filing date of U.S. Provisional Patent Application No. 63/466,190 field on May 12, 2023, the disclosure of which is hereby incorporated by reference herein in its entirety.

### STATEMENT REGARDING FEDERALLY SPONSORED RESEARCH

Not applicable.

### BACKGROUND

The disclosure relates, in general, to the enzymatic conversion of methylated nucleic acids in order to distinguish between methylated and unmethylated cytosines in DNA and, more particularly, to improved methods and compositions for enzymatic methylation sequencing.

Methylation of DNA, as one of the major epigenetic mechanisms, plays a significant role in a variety of biological processes including, for example, regulating gene expression, organism development, X chromosome inactivation and genetic imprinting in vertebrates. Detecting the methylation status and changes in methylation patterns in DNA is important for many clinical applications including examination of circulating tumor DNA for tissue of origin and disease state analysis. Historically, nucleobase level detection of modified cytosines in DNA has been achieved by deaminating unmodified/unmethylated cytosines via bisulfite treatment followed by amplification via polymerase chain reaction (PCR). The bisulfite treatment reaction is highly efficient; however, the method has several drawbacks, including the need for i) a large amount of input DNA due to the harsh nature of the chemical treatment, and ii) long incubation times in order to achieve sufficient conversion of unmethylated cytosines. For liquid biopsy applications that are limited by the amount of available sample DNA for testing, bisulfite treatment methods are hampered by low unique molecule recovery and significant loss of useful information about methylation status of the input DNA.

An enzymatic version of cytosine deamination has recently been developed, termed “enzymatic methylation sequencing” or “EM-seq”, which may overcome some of the drawbacks of the chemical degradation of DNA from bisulfite treatments (see Vaisvila et al., Genome Res. 2021. 31: 1280-1289). The method involves tet methylcytosine dioxygenase 2 (TET2 or TET) oxidation and T4-phage beta-glucosyltransferase (T4-βGT) glucosylation to protect the 5-methylcytosines (5mC) and 5-hydroxymethylcytosines (5hmC) from deamination. Subsequently, apolipoprotein B mRNA editing enzyme catalytic subunit 3A (APOBEC3A or A3A) deaminates all unmodified cytosines to uracils. This enzymatic version of cytosine conversion for methylation detection by sequencing has been demonstrated to recover more unique molecules and provide better coverage uniformity across the input DNA. However, EM-seq may not be suitable for low sample input applications (i.e., nanogram to pictogram amounts of input DNA) due to insufficient unique molecule recovery and deamination reaction efficiency.

Additional challenges observed with methylation sequencing workflows (including both EM-seq and bisulfite sequencing), relate to library preparation. Specifically, current methylation sequencing workflows include one or more library preparation steps that prepare the sample for sequencing and one or more conversion steps that enable nucleobase resolution methylation detection. In general, a first step of library preparation is to repair and blunt the ends of double stranded DNA (dsDNA) to allow efficient ligation of nucleic acid adapters for sequencing. During this step, if nicks are present on the dsDNA, the process of nick translation effected by the repair enzymes present in this step can eliminate a substantial amount of methylation signal and result in bias of unmethylated cytosines towards the 3′end of the molecule. This methylation bias or M-bias is widely observed in samples such as cell-free DNA (cfDNA), where the dsDNA molecules contain various nicks due, for example, to endonucleases present in the plasma or oxidative processes.

For at least the forgoing reasons, there is a need for improved methods for distinguishing methylated cytosines from unmethylated cytosines in DNA samples for a variety of applications.

### SUMMARY

The present invention overcomes the aforementioned drawbacks by providing methods for the enzymatic conversion of methylated nucleic acids for sequencing.

In accordance with one embodiment of the present disclosure, a method includes contacting a first enzyme possessing ligase activity with a double-stranded DNA (dsDNA) comprising at least one nick, at least one cytosine, and at least one methylcytosine selected from 5-methylcytosine and 5-hydroxymethylcytosine, thereby repairing the at least one nick, converting the at least one methylcytosine to a modified methylcytosine capable of base-pairing with a guanine, and converting the at least one cytosine to uracil.

In one aspect, the modified methylcytosine is selected from 5-(β-glucosyloxymethyl)cytosine and 5-carboxycytosine (5caC).

In another aspect, the at least one methylcytosine is converted with a second enzyme to the modified methylcytosine.

In another aspect, the second enzyme is selected from a methylcytosine dioxygenase and a beta-glucosyltransferase.

In another aspect, the at least one cytosine is converted with a third enzyme to uracil.

In another aspect, the third enzyme is apolipoprotein B mRNA editing enzyme catalytic subunit 3A (APOBEC3A).

In another aspect, the first enzyme is a Taq ligase.

In another aspect, the method further includes ligating sequencing adaptors to the dsDNA.

In another aspect, the method further includes contacting the nick-repaired dsDNA with a fourth enzyme comprising at least one of DNA end-repair activity and A-tailing activity.

In another aspect, the methylcytosine dioxygenase is TET methylcytosine dioxygenase 2 and wherein the beta-glucosyltransferase is T4-phage beta-glucosyltransferase.

In accordance with another embodiment of the present disclosure, a method includes contacting a Taq ligase with a double-stranded DNA (dsDNA) comprising at least one nick, at least one cytosine, and at least one methylcytosine selected from 5-methylcytosine and 5-hydroxymethylcytosine, thereby repairing the at least one nick, enzymatically converting with a TET methylcytosine dioxygenase 2 (TET) the at least one methylcytosine to one of 5-hydroxmethylcytosine and 5-carboxycytosine, enzymatically converting with a T4-phage beta-glucosyltransferase (T4-BGT) 5-hydroxmethylcytosine to 5-(β-glucosyloxymethyl)cytosine, and enzymatically converting with an apolipoprotein B mRNA editing enzyme catalytic subunit 3A (APOBEC3A) the at least one cytosine to uracil.

In one aspect, the method further includes ligating sequencing adaptors to the dsDNA.

In another aspect, the method further includes contacting the nick-repaired dsDNA with an enzyme comprising at least one of DNA end-repair activity and A-tailing activity.

In another aspect, the method further includes contacting the dsDNA with an apurinic/apyrimidinic endonuclease.

In accordance with another embodiment of the present disclosure, a method includes, contacting a first enzyme possessing methyltransferase activity with a double-stranded DNA (dsDNA) comprising at least one hemimethylated CpG site, at least one additional cytosine and at least one methylcytosine selected from 5-methylcytosine and 5-hydroxymethylcytosine, wherein the at least one additional cytosine remains unmethylated, converting the at least one methylcytosine to a modified methylcytosine capable of base-pairing with a guanine; and converting the at least one additional cytosine to uracil.

In one aspect, the modified methylcytosine is selected from 5-(β-glucosyloxymethyl)cytosine and 5-carboxycytosine (5caC).

In another aspect, the at least one methylcytosine is converted with a second enzyme to the modified methylcytosine.

In another aspect, the second enzyme is selected from a methylcytosine dioxygenase and a beta-glucosyltransferase.

In another aspect, the at least one additional cytosine is converted with a third enzyme to uracil.

In another aspect, the third enzyme is apolipoprotein B mRNA editing enzyme catalytic subunit 3A (APOBEC3A).

In another aspect, the first enzyme is Human DNA (cytosine-5) Methyltransferase.

In another aspect, the method further includes ligating sequencing adaptors to the dsDNA.

In another aspect, the method further includes contacting the dsDNA with a fourth enzyme comprising at least one of DNA end-repair activity and A-tailing activity.

In another aspect, the methylcytosine dioxygenase is TET methylcytosine dioxygenase 2 and wherein the beta-glucosyltransferase is T4-phage beta-glucosyltransferase.

In accordance with another embodiment of the present disclosure, a method includes ligating an adapter to a double-stranded DNA (dsDNA) comprising at least one cytosine and at least one methylcytosine selected from 5-methylcytosine and 5-hydroxymethylcytosine, the adapter comprising a nucleic acid sequencing comprising at least one methylcytosine, wherein each methylcytosine present in the adapter is a 5-hydroxymethylcytosine, converting the at least one methylcytosine present in the dsDNA and the at least one methylcytosine present in the adapter to a modified methylcytosine capable of base-pairing with a guanine; and converting the at least one additional cytosine to uracil.

In one aspect, the modified methylcytosine is selected from 5-(β-glucosyloxymethyl) cytosine and 5-carboxycytosine (5caC).

In another aspect, the at least one methylcytosine is converted with a first enzyme to the modified methylcytosine.

In another aspect, the first enzyme is selected from a methylcytosine dioxygenase and a beta-glucosyltransferase.

In another aspect, the at least one cytosine is converted with a second enzyme to uracil.

In another aspect, the second enzyme is apolipoprotein B mRNA editing enzyme catalytic subunit 3A (APOBEC3A).

In another aspect, the method further includes contacting the dsDNA with a third enzyme comprising at least one of DNA end-repair activity and A-tailing activity.

In another aspect, the methylcytosine dioxygenase is TET methylcytosine dioxygenase 2 and wherein the beta-glucosyltransferase is T4-phage beta-glucosyltransferase.

In accordance with another embodiment of the present disclosure, a method includes, ligating an adapter to a double-stranded DNA (dsDNA) comprising at least one cytosine and at least one methylcytosine selected from 5-methylcytosine and 5-hydroxymethylcytosine, the adapter comprising a nucleic acid sequencing comprising at least one modified methylcytosine, wherein each modified methylcytosine present in the adapter is a 5-(β-glucosyloxymethyl)cytosine, converting the at least one methylcytosine present in the dsDNA to a modified methylcytosine capable of base-pairing with a guanine, and converting the at least one additional cytosine to uracil.

In one aspect, the modified methylcytosine present in the dsDNA is selected from 5-(β-glucosyloxymethyl) cytosine and 5-carboxycytosine (5acC).

In another aspect, the at least one methylcytosine in the dsDNA is converted with a first enzyme to the modified methylcytosine.

In another aspect, the first enzyme is selected from a methylcytosine dioxygenase and a beta-glucosyltransferase.

In another aspect, the at least one cytosine is converted with a second enzyme to uracil.

In another aspect, the second enzyme is apolipoprotein B mRNA editing enzyme catalytic subunit 3A (APOBEC3A).

In another aspect, the method further includes contacting the dsDNA with a third enzyme comprising at least one of DNA end-repair activity and A-tailing activity.

In another aspect, the methylcytosine dioxygenase is TET methylcytosine dioxygenase 2 and wherein the beta-glucosyltransferase is T4-phage beta-glucosyltransferase.

In accordance with another embodiment of the present disclosure, an adaptor for methyl-sequencing includes a first nucleic acid having a 3′ end complementary to a 5′ end of a second nucleic acid, thereby forming a complementary region, the complementary region comprising at least one modified methylcytosine, wherein each modified methylcytosine present in the adapter is a 5-(β-glucosyloxymethyl)cytosine.

In accordance with another embodiment of the present disclosure, an adaptor for methyl-sequencing includes a first nucleic acid having a 3′ end complementary to a 5′ end of a second nucleic acid, thereby forming a complementary region, the complementary region comprising at least one modified methylcytosine, wherein each modified methylcytosine present in the adapter is a 5-hydroxymethylcytosine.

In accordance with another embodiment of the present disclosure, a method includes combining i) a double-stranded DNA (dsDNA) comprising at least one cytosine, and at least one methylcytosine selected from 5-methylcytosine and 5-hydroxymethylcytosine, ii) a cytidine deaminase and iii) at least one of a single-strand DNA-binding protein and a helicase, converting the at least one methylcytosine to a modified methylcytosine capable of base-pairing with a guanine, and converting with the cytidine deaminase the at least one cytosine to uracil.

In one aspect, the modified methylcytosine is selected from 5-(β-glucosyloxymethyl) cytosine and 5-carboxycytosine (5caC).

In another aspect, the at least one methylcytosine is converted to the modified methylcytosine with at least one of a methylcytosine dioxygenase and a beta-glucosyltransferase.

In another aspect, the cytidine deaminase is apolipoprotein B mRNA editing enzyme catalytic subunit 3A (APOBEC3A).

In another aspect, the method further includes ligating sequencing adaptors to the dsDNA.

In another aspect, the method further includes contacting the dsDNA with an enzyme having at least one of DNA end-repair activity and A-tailing activity.

In another aspect, the methylcytosine dioxygenase is TET methylcytosine dioxygenase 2 and wherein the beta-glucosyltransferase is T4-phage beta-glucosyltransferase.

In accordance with another embodiment of the present disclosure, a method includes combining i) a double-stranded DNA (dsDNA) comprising at least one cytosine, and at least one methylcytosine selected from 5-methylcytosine and 5-hydroxymethylcytosine, ii) a cytidine deaminase and iii) at least one of a single-strand DNA-binding protein and a helicase, enzymatically converting with a TET methylcytosine dioxygenase 2 (TET) the at least one methylcytosine to one of 5-hydroxmethylcytosine and 5-carboxycytosine, enzymatically converting with a T4-phage beta-glucosyltransferase (T4-BGT) 5-hydroxmethylcytosine to 5-(β-glucosyloxymethyl)cytosine, and enzymatically converting with an apolipoprotein B mRNA editing enzyme catalytic subunit 3A (APOBEC3A) the at least one cytosine to uracil.

In one aspect, the method further includes ligating sequencing adaptors to the dsDNA.

In another aspect, the method further includes contacting the dsDNA with an enzyme having at least one of DNA end-repair activity and A-tailing activity.

In accordance with another embodiment of the present disclosure, a composition includes a double-stranded DNA (dsDNA) comprising at least one cytosine, and at least one modified cytosine selected from 5-carboxycytosine and 5-(β-glucosyloxymethyl) cytosine, a buffer, and a cytidine deaminase, wherein the amount of the dsDNA is from about 1 picogram to about 10 ng, wherein the concentration of the cytidine deaminase is from about 0.05 uM to about 0.5 uM, and wherein the total volume of the composition is less than about 100 uL.

In accordance with another embodiment of the present disclosure, a method includes providing a first composition comprising a double-stranded DNA (dsDNA) comprising at least one cytosine, and at least one methylcytosine selected from 5-methylcytosine and 5-hydroxymethylcytosine, enzymatically converting with a TET methylcytosine dioxygenase 2 (TET) the at least one methylcytosine to one of 5-hydroxmethylcytosine and 5-carboxycytosine, thereby forming a first dsDNA product, enzymatically converting with a T4-phage beta-glucosyltransferase (T4-BGT) 5-hydroxmethylcytosine in the first dsDNA product to 5-(β-glucosyloxymethyl)cytosine, thereby forming a second dsDNA product, enzymatically converting with an apolipoprotein B mRNA editing enzyme catalytic subunit 3A (APOBEC3A) the at least one cytosine in the second dsDNA product to uracil, thereby forming a methyl-sequencing product composition comprising the APOBEC3A and a third dsDNA product, combining the methyl-sequencing product composition with an amplification composition, and amplifying by polymerase chain reaction the third dsDNA product, wherein the amplifying occurs in the presence of the APOBEC3A.

In one aspect, the each of the aforementioned compositions and methods can further include an engineered DNA ligase for ligating an adapter to dsDNA.

In accordance with another embodiment of the present disclosure, a method includes combining a first enzyme possessing ligase activity, a second enzyme possessing 5′ to 3′ polymerase activity, the second enzyme lacking 5′ to 3′ exonuclease activity, a third enzyme possessing apurinic/apyrimidinic endonuclease activity, a double-stranded DNA (dsDNA) comprising at least one single-stranded discontinuity selected from a nick and a gap, at least one cytosine, and at least one methylcytosine selected from 5-methylcytosine and 5-hydroxymethylcytosine, and a plurality of deoxynucleoside triphosphates (dNTPs). The method further includes repairing the at least one discontinuity in the dsDNA, converting the at least one methylcytosine to a modified methylcytosine capable of base-pairing with a guanine, and converting the at least one cytosine to uracil.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is an example of a first method 100 and a second method 200 for detection of DNA methylation by enzymatic methylation sequencing according to the present disclosure.

FIG. 2a is a schematic illustration of a process for the enzymatic conversion of methylated DNA for sequencing. Above the dotted line, a plurality of enzymatic methylation sequencing reactions is illustrated for conversion of both unmethylated and methylated cytosine bases. Arrows indicate enzymatic conversion of one molecule (shown in a rounded rectangle) to another molecule by the enzyme listed adjacent to the arrow. An “X” over an arrow indicates that the depicted enzyme exhibits low or no activity for conversion of the indicated molecules. Below the dotted line, the nucleobase detected by sequencing is shown for a corresponding product of the EM-seq conversion reactions as indicated by the dashed arrows (e.g., uracil is ultimately detected as thymine by sequencing).

FIG. 2b is an illustration of a selection of methylated and unmethylated nucleobases from FIG. 2a. From left to right, the illustrated molecules include cytosine, 5-methylcytosine (5mC) and 5-hydroxymethylcytosine (5hmC), 5-formylcytosine (5fC), 5-carboxycytosine (5caC), and 5-(β-glucosyloxymethyl)cytosine (5gmC).

FIG. 3 is a bar plot illustrating the effect of the selection of ligase (engineered DNA ligase vs. wild type T4 DNA ligase) on the average rate of CpG modification of human cfDNA (“primary”) in reference to an unmethylated lambda spike in control (“lambda”) and methylated pUC19 plasmid DNA spike in control (“pUC19”). Data is shown for 3 different cfDNA samples with two replicates for each sample in the format “[sample number]-[replicate number]”.

FIG. 4 is a bar plot illustrating the effect of the selection of ligase (engineered DNA ligase vs. wild type T4 DNA ligase) on reduction in duplication rate as a measure of the recovery of unique molecules from a human cfDNA sample. Data is shown for 3 different cfDNA samples with two replicates for each sample in the format “[sample number]-[replicate number]”.

FIG. 5 is a plot illustrating the effect of the addition of Taq ligase prior to end repair and A-tailing on methylation signal at the 3′-end of a sample DNA molecule as determined by nucleic acid sequencing.

FIG. 6 is a bar plot illustrating conversion efficiency of EM-seq reactions for DNA samples ligated to adapters in which each modified cytosine is 5mC (lambda mC) or in which each modified cytosine is 5hmC (primary hmC).

FIG. 7 is a pair of plots illustrating the effect of the addition of Taq DNA ligase and methyltransferase DNMT5 from Cryptococcus neoformans prior to end repair and A-tailing on methylation signal at the 3′-end of a sample DNA molecule as determined by nucleic acid sequencing.

### DETAILED DESCRIPTION

### I. Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level, and/or form of one is correlated with that of the other. For example, a particular entity (e.g., polypeptide, genetic signature, metabolite, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises or consists of an organism, such as an animal or human. In some embodiments, a biological sample is comprises or consists of biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom, etc. In some embodiments, a biological sample is comprises or consists of cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Comprising: A composition or method described herein as “comprising” one or more named elements or steps is open-ended, meaning that the named elements or steps are essential, but other elements or steps may be added within the scope of the composition or method. It is to be understood that composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Designed: As used herein, the term “designed” refers to an agent (i) whose structure is or was selected by the hand of man; (ii) that is produced by a process requiring the hand of man; and/or (iii) that is distinct from natural substances and other known agents.

Determine: Those of ordinary skill in the art, reading the present specification, will appreciate that “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM 120 weight residue table, a gap length penalty of 12 and a gap penalty of 4. The percent identity between two nucleotide sequences can, alternatively, be determined using the GAP program in the GCG software package using an NWSgapdna.CMP matrix.

Sample: As used herein, the term “sample” refers to a substance that is or contains a composition of interest for qualitative and or quantitative assessment. In some embodiments, a sample is a biological sample (i.e., comes from a living thing (e.g., cell or organism). In some embodiments, a sample is from a geological, aquatic, astronomical, or agricultural source. In some embodiments, a source of interest comprises or consists of an organism, such as an animal or human. In some embodiments, a sample for forensic analysis is or comprises biological tissue, biological fluid, organic or non-organic matter such as, e.g., clothing, dirt, plastic, water. In some embodiments, an agricultural sample, comprises or consists of organic matter such as leaves, petals, bark, wood, seeds, plants, fruit, etc.

Specificity: As used herein, the term “specificity” means the preference of an enzyme for a specific substrate.

Selectivity: As used herein, the term “selectivity” means the preference of an enzyme for one specific substrate over another.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Synthetic: As used herein, the word “synthetic” means produced by the hand of man, and therefore in a form that does not exist in nature, either because it has a structure that does not exist in nature, or because it is either associated with one or more other components, with which it is not associated in nature, or not associated with one or more other components with which it is associated in nature.

Methylated: As used herein, the term “methylated” means a cytosine having a methyl or hydroxymethyl group at the C-5 position of the cytosine ring of DNA. A methylated DNA is a DNA having at least one methylated cytosine. Methylated cytosine in DNA can naturally include 5-methylcytosine (5mC) and 5-hydroxymethylcytosine (5hmC), 5-formylcytosine (5fC), and 5-carboxycytosine (5caC); however, 5mC may be present in methylated DNA in a significantly greater abundance than 5hmC, which, in turn, may be present in methylated DNA in a significantly greater abundance than 5fC and 5caC.

Unmethylated: As used herein, the term “unmethylated” or “non-methylated” means a cytosine lacking a methyl or hydroxymethyl group at the C-5 position of the cytosine ring of DNA. An unmethylated DNA is a DNA lacking methylated cytosine.

Methylation signal: As used herein, the term “methylation signal” means a signal measured as a proxy for cytosine methylation. In embodiments of the present disclosure, methylation signal can include signal assigned as cytosine as determined by nucleic acid sequencing.

End Repair: As used herein, the term “end repair” refers to methods for repairing DNA (e.g., fragmented or damaged DNA or DNA molecules that are incompatible with other DNA molecules). In some embodiments, the process involves two functions: 1) conversion of double-stranded DNA with overhangs to double-stranded DNA without overhangs by an enzyme such as T4 DNA polymerase and/or Klenow fragment; and 2) addition of a phosphate group to the 5′ ends of DNA (single-or double-stranded), by an enzyme such as polynucleotide kinase.

A-Tailing: As used herein, the term “A-tailing” refers to the addition of a single deoxyadenosine residue to the end of a blunt-ended double-stranded DNA fragment to form a 3′ deoxyadenosine single-base overhang. A-tailed fragments are not compatible for self-ligation (i.e., self-circularization and concatenation of the DNA), but they are compatible with 3′ deoxythymidine-overhangs such as those present on adapters.

Conversion: As used herein, “conversion” refers to the enzymatic conversion (or biotransformation) of substrate(s) to the corresponding product(s). “Percent conversion” refers to the percent of the substrate that is converted to the product within a period of time under specified conditions. Thus, the “enzymatic activity” or “activity” of an enzyme can be expressed as “percent conversion” of the substrate to the product in a specific period of time.

### II. Detailed Description of Certain Embodiments

As also discussed above, in various situations it may be useful to provide a method for conversion of methylated nucleic acids in order to distinguish between methylated and unmethylated cytosines in DNA and, more particularly, to improved methods and compositions for methylation sequencing (including EM-seq and bisulfite sequencing). In one aspect, methylation sequencing may not be suitable for low sample input applications (i.e., nanogram to picogram amounts of input DNA) due to insufficient unique molecule recovery and deamination reaction efficiency. In another aspect, current methylation sequencing methods may be subject to methylation bias in samples such as cfDNA due to the presence of DNA damage caused by endonucleases, oxidative processes, and the like.

These and other challenges may be overcome with compositions and methods for enzymatic conversion of methylated nucleic acids for sequencing according to the present disclosure. In one aspect, the present disclosure provides for improved recovery of methylation signal (i.e., accurate detection of methylated and unmethylated cytosines by sequencing). Improvements are achieved, at least in part, through a variety of approaches, including repair of nicks present in sample DNA prior to select library preparation steps, restoration of methylation signal in the case of hemimethylated dsDNA, use of nucleic acid adaptors comprising or consisting of methylcytosines selected from 5mC, 5hmC and of 5-(β-glucosyloxymethyl)cytosine (5gmC), use of helicase, ssDNA binding proteins, or a combination thereof, and improved compositions for library preparation.

The present disclosure is, at least in part, based on the surprising discovery that the selection of a DNA ligase for attachment of nucleic acid adaptors has a significant impact on the downstream detection of methylation. In one aspect, the selection of a DNA ligase for adapter ligation improved conversion efficiency (i.e., the efficiency of adapter ligated target product) at very low sample input amounts, and further improved ligation specificity which results in a decreased sensitivity to adapter concentrations and a reduced observation of adapter dimer formation. Moreover, the selection of specific DNA ligases enabled the reduction of the ligation reaction time from 16 hours for a standard DNA ligase to about 5 minutes with a ligase according to the present disclosure.

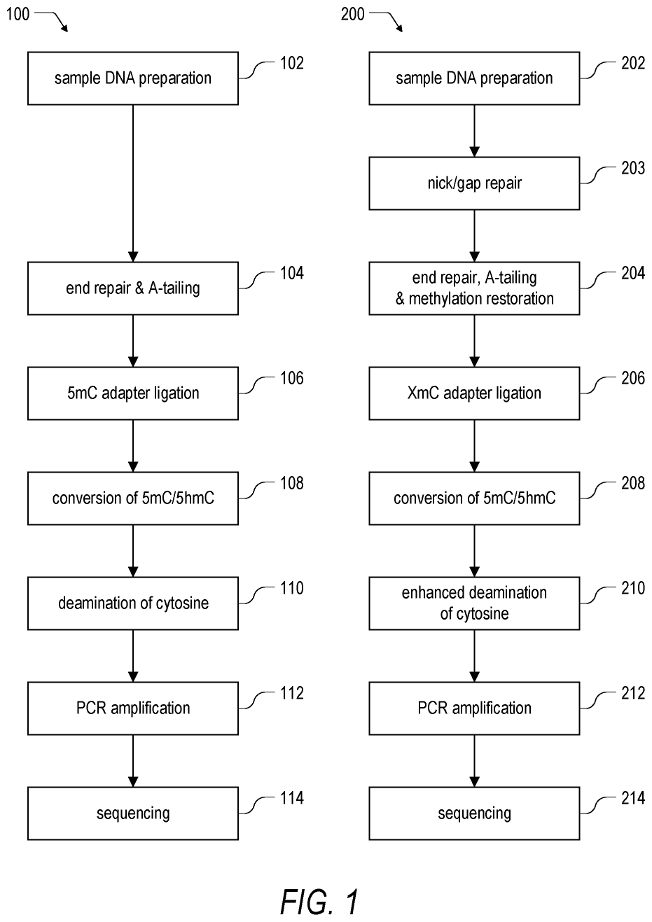

Turning to FIG. 1, an embodiment of a method 100 for enzymatic methylation (EM) sequencing is illustrated. In one aspect, the method 100 represents a current approach known in the art for enzymatic conversion of methylated DNA samples for detection of methylation using next generation sequencing technologies, such as flow-cell based sequencing by synthesis approaches. In general, detection of methylation, and specifically, 5mC and 5hmC, which can occur naturally in DNA, is achieved by selective deamination of unmethylated cytosine to uracil, leaving 5mC and 5hmC (and derivatives thereof) intact. Amplification of the product of the selective deamination step by PCR converts each uracil to thymine, whereas 5hmC and 5mC (and derivatives thereof) are converted to cytosine. As in the case of bisulfite sequencing, the amplified DNA retains only methylated cytosines, yielding single-nucleotide resolution information about the methylation status of a the DNA which can be elucidated using a variety of known informatics approaches.

According to a step 102 of the method 100, sample DNA is prepared. Preparation of sample DNA can include collection of DNA or materials containing DNA such as whole blood, plasma, serum, tissue (including formalin fixed paraffin embedded or FFPE tissue), or the like. The step 102 can further include recovery of the DNA, if applicable, from a blood or tissue sample, or other DNA containing sample. In one aspect, the DNA is cfDNA. In another aspect, the DNA is circulating tumor DNA (ctDNA). In another aspect, the sample DNA is double stranded DNA (dsDNA). In another aspect, the DNA is sheared or otherwise fragmented to provide a uniform size or desired size distribution with respect to the length in nucleotides of the DNA. In another aspect, the DNA is purified using one or more purifications methods to obtain isolated DNA. The step 102 can include any other necessary steps to prepare the sample DNA for downstream steps of the method 100. In one aspect, the sample DNA includes DNA damage, including but not limited to single stranded nicks, doubled stranded breaks, hemimethylation (i.e., partial methylation at a CpG site on only one strand of a dsDNA), and the like. For clarity, a CpG site or CG site is defined as a region of DNA where a cytosine is followed by a guanine in a linear sequence of bases in the 5- to 3′ direction. In another aspect, the sample DNA includes cytosine bases, at least a portion of which are methylated. In another aspect, the methylated cytosine bases include at least one of 5mC and 5hmC.

In a step 104, the DNA sample is treated with one or more enzymes to convert fragmented DNA to repaired DNA having 5′ phosphorylated, 3′ dA-tailed ends (also known as end repair and A-tailing). Suitable enzymes include DNA polymerases having 3′ exonuclease activity, such as a Taq DNA polymerase I derived from Thermus aquaticus, T4 DNA polymerase, T4 polynucleotide kinase, and Klenow fragment. In one aspect, the sample DNA product of the step 104 can be purified away from any enzymes, salts, buffers or other reagents used in the step 104. In one example, a suitable combination of enzymes for end repair and A-tailing includes a polynucleotide kinase, a first DNA polymerase, and a second DNA polymerase. In one aspect, the polynucleotide kinase has activity for 5′ phosphorylation and removal of 3′ phosphoryl groups (e.g., T4 polynucleotide kinase). In another aspect, the first DNA polymerase has activity for gap filling, and removal of 3′ overhangs or fill-in of 5′ overhangs to form blunt ends, but lacks strand displacement and 5′ to 3′ exonuclease activity (e.g., T4 DNA polymerase). In another aspect, the second DNA polymerase has activity for at least the addition of 3′dA, also known as A-tailing (e.g., Taq DNA polymerase).

In a step 106, nucleic acid adapters may be added to enable downstream workflows or other processing steps. In one aspect, the adapters are selected to enable downstream sequencing steps. In another aspect, the adapters are selected to enable downstream amplification steps. In general, addition of adapters to the repaired DNA from the step 104 includes an enzymatic ligation step. Accordingly, the adapters can include 5′ phosphorylated, 3′ dA-tailed ends compatible with the sample DNA resulting from the step 104; however, other approaches known in the art can also be applied. Example adapters include Y-adapters, dumbbell adapters, blunt adapters, overhang adapters, single stranded adapters and the like. Adapters may further include sample barcode/identification sequences to distinguish one sample DNA from another, unique molecular identification sequences to distinguish between individual DNA molecules, and the like. For EM-seq, bisulfite sequencing, and other methylation detection approaches, it may be useful to provide adapters in which some or all of the cytosines present in the sample are converted or otherwise provided as 5mC. In one aspect, the sample DNA product of the step 106 can be purified away from any enzymes, salts, buffers or other reagents used in the step 106.

In a step 108, the repaired DNA including the 5mC adapters applied thereto is treated to convert the 5mC and 5hmC present in the DNA to a modified methylcytosine. As defined herein, a modified methylcytosine is a derivative or modified structure of 5mC or 5hmC that is not a substrate for the cytidine deaminase. It should be appreciated that the purpose of the step 108 is to convert the 5mC and 5hmC to a derivative or modified structure that is not a substrate for the cytidine deaminase selected for use in the step 110. Accordingly, the converted 5mC and 5hmC are protected from deamination in the step 110. Moreover, the converted 5mC and 5hmC are preferably capable of effectively base pairing with a guanine base during PCR in the step 112. As discussed above, the outcome of the method 100 is to provide amplified DNA that retains only methylated cytosines, yielding single-nucleotide resolution information about the methylation status of the DNA for downstream analysis.

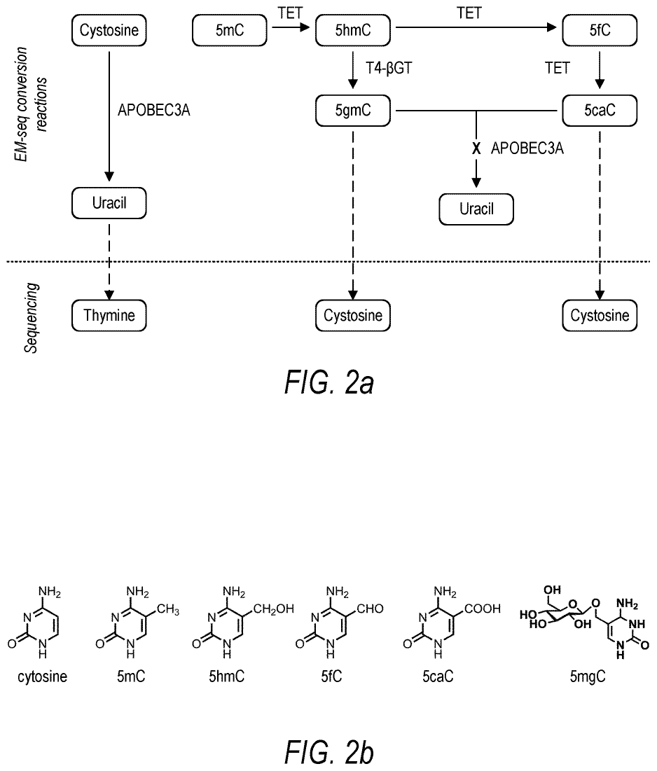

The step 108 can include one or more enzymes to selectively convert 5mC and 5hmC while leaving cytosine intact or otherwise unmodified. Example enzymes known for use in EM-seq include methylcytosine dioxygenase and a beta-glucosyltransferase. One example methylcytosine dioxygenase is TET. One example beta-glucosyltransferase is T4-BGT. With reference to FIGS. 2a and 2b, TET can enzymatically catalyze the formation of 5hmC from 5mC. Moreover, TET can further act on 5hmC, yielding 5-formylcytosine (5fC), which can in turn be converted by TET to 5-carboxycytosine (5caC). In one aspect, T4-BGT can enzymatically catalyze the formation of 5gmC from 5hmC. In another aspect, it may be useful to further convert 5hmC or 5fC to a corresponding derivative with TET, T4-BGT or another enzyme for the reason that a cytidine deaminase may be selected with little to no activity on either 5gmC or 5caC as will be discussed with respect to the step 110. By contrast, cytidine deaminase may have some activity on 5hmC and 5fC, which as discussed above, may be desirable to avoid in order to achieve selective deamination of only cytosine in or to distinguish from methylated cytosine in downstream sequencing applications. In one aspect, the step 108 ideally involves complete conversion of each 5mC and 5hmC present in the sample DNA. However, depending on the enzyme selected, the reaction conditions, and other factors, it is likely that less than 100% percent conversion of each 5mC and 5hmC is reasonably achieved. Preferably, at least 70% conversion is achieved. More preferably at least 80%, 90%, 91%, 92%, 93%, 94%, 95%, 96%, 97%, 98%, 99% or at least 99.5% conversion is achieved. In one aspect, the sample DNA product of the step 108 can be purified away from any enzymes, salts, buffers or other reagents used in the step 108.

In a step 110, the DNA product of the step 108, including the converted 5mC and converted 5hmC, is treated with a cytidine deaminase in order to selectively convert unmethylated or otherwise unmodified cytosine bases present in the sample DNA to uracil. As defined herein, a cytidine deaminase is any enzyme capable of converting a cytosine base present in a nucleic acid to uracil. Accordingly, a cytidine deaminase may be effective for converting cytosine bases in both cytidine (e.g., in RNA) and deoxycytidine (e.g., in DNA).

As discussed above, the step 110 is carried out to chemically/structurally differentiate cytosine bases from methylated cytosine bases. By selectively deaminating only the unmethylated cytosine bases to uracil bases, PCR can be used to amplify the resulting product. In this case, when using only the deoxynucleoside triphosphates (dNTPs) adenine cytosine, guanine and thymine (i.e., dATP, dCTP, dGTP, and dTTP, respectively), any uracil bases will result in thymine bases, while methylated cytosine bases in any of the aforementioned modified forms will result in cytosine bases. In one aspect, the cytidine deaminase is APOBEC3A; however, other cytidine deaminases can be selected as will be appreciated by one of ordinary skill in the art. In one aspect, the step 108 ideally involves complete conversion of each unmethylated cytosine base present in the sample DNA. However, depending on the enzyme selected, the reaction conditions, and other factors, it is likely that less than 100% percent conversion of each cytosine base is reasonably achieved. Preferably, at least 70% conversion is achieved. More preferably at least 80%, 90%, 91%, 92%, 93%, 94%, 95%, 96%, 97%, 98%, 99% or at least 99.5% conversion is achieved. Moreover, ideally cytosine deamination is 100% selective for cytosine over methylated cytosines, including any converted methylcytosines (e.g., 5hmC, 5fC, 5gmC, and 5caC). By 100% selective, it is meant that methylcytosine bases present in the sample DNA are not a substrate for the cytidine deaminase. However, depending on the enzyme selected, the reaction conditions, and the like, it is possible that the cytidine deaminase exhibits some activity on methylcytosines present in the sample DNA in the step 110 (i.e., the cytidine deaminase exhibits less than 100% selectivity). Preferably, at least 70% selectivity is achieved. More preferably at least 80%, 90%, 91%, 92%, 93%, 94%, 95%, 96%, 97%, 98%, 99% or at least 99.5% selectivity is achieved. In one aspect, the aforementioned selectivity is achieved with respect to at least a one methylcytosine structure selected from 5hmC, 5fC, 5gmC, and 5caC. In one aspect, the aforementioned selectivity is achieved with respect to at least a one methylcytosine structure selected from 5gmC and 5caC. In one aspect, the sample DNA product of the step 110 can be purified away from any enzymes, salts, buffers or other reagents used in the step 110.

In a step 112, the sample DNA resulting from the step 110 is amplified using PCR. In one aspect, the sample DNA resulting from the step 110 ideally includes only methylated cytosine bases and no unmethylated cytosine bases, as each unmethylated cytosine base was preferably converted to uracil in the step 110. In another aspect, the methylated cytosine bases include 5gmC and 5caC. The step 112 can include PCR amplification with dNTPs consisting of dATP, dCTP, dGTP and dTTP. Primers can be selected that are complementary to adapter nucleic acid sequences attached to the sample DNA in the step 106. The primers can further include sample identification sequences or unique molecular identification sequences as discussed above. In one aspect, the sample DNA product of the step 112 can be purified away from any enzymes, salts, buffers or other reagents used in the step 112. In one aspect, the product of the step 112 may be referred to as a library. The term “library” may also be reasonably applied to the products of any of the preceding steps 104, 106, 108 and 110.

In a step 114, the sequences of the products of the PCR amplification step 112 are determined. In one aspect, any suitable DNA sequencing technology may be selected to determine the nucleic acid sequences of interest. For example, sequencing by synthesis may be used. The data collected from sequencing determines the identity of each base in a nucleic acid sequence present in the sequenced library. As discussed above, each cytosine base detected by sequencing can be interpreted as a methylcytosine base-likely 5mC or 5hmC-in the original sample DNA sequence, whereas each thymine base detected can be interpreted as a thymine or cytosine in the original sample DNA sequence. Using known informatics tools, the assignment of thymine or cytosine can be made or predicted with respect to the sequence identify of the original sample DNA.

The method 100, as described, is generally useful for EM-seq. However, the present disclosure provides for several novel improvements and modifications to the method 100. With continued reference to FIG. 1, a method 200 illustrates at least some of the disclosed improvements to an EM-seq workflow. In a step 202, a sample DNA input is prepared. In some embodiments, the step 202 can be similar to, or the same as, the step 102 of the method 100. In other embodiments, the step 202 can be modified to provide the sample DNA in a format compatible with subsequent steps in the method 200.

In a step 203 of the method 200, potential loss of methylation signal is mitigated through the repair of either or both of nicks and gaps in a dsDNA that may be present in the sample DNA. In general, dsDNA contains nicks (i.e., discontinuities in the dsDNA molecule where there is no phosphodiester bond between adjacent nucleotides of one strand of the dsDNA). By contrast, gaps in dsDNA are discontinuities in the dsDNA molecule where one or more contiguous nucleotides are missing from one strand of the dsDNA. Nicks and gaps in dsDNA can provide replication initiation sites for DNA polymerase, enabling a process known as nick translation. When nick translation occurs, a DNA polymerase elongates the 3′ hydroxyl terminus of the nick or gap site, removing nucleotides by 5′ to 3′ exonuclease activity, and replacing them with dNTPs. Accordingly, the strand of the dsDNA located 3′ of the nick, including any methylated or otherwise modified bases, is absent from the product of nick translation, and the corresponding methylation signal is lost. The step 203 contemplates repairing of nicks, gaps, or both to mitigate the potentially detrimental effects of nick translation on the detection of methylation. It will be appreciated that not all DNA polymerases possess 5′ to 3′ exonuclease activity or the capability to otherwise catalyze nick translation. Accordingly, the step 203 may include one or more DNA polymerases having activity for gap filling without strand displacement, removal of 3′ overhangs or fill-in of 5′ overhangs to form blunt ends, or the like, as long as such DNA polymerases lack activity for nick translation.

In some embodiments of the step 203, nicks are repaired with an enzyme such as a ligase. In one aspect, the ligase possesses dsDNA nick repair activity. In one example, the ligase is a DNA ligase derived from a Thermus species, such as Taq DNA Ligase from Thermus thermophilus HB8. In one aspect, a ligase possessing dsDNA nick repair activity catalyzes the formation of a phosphodiester bond between the 5′-phosphate and the 3′-hydroxyl of two adjacent DNA strands in a nicked dsDNA. In particular, a ligase possessing dsDNA nick repair activity should preferentially catalyze bond formation when the strands to be ligated are hybridized and accurately paired, with no gap, to a complementary DNA strand. In another aspect, a ligase possessing dsDNA nick repair activity should have limited or no activity for ligation of blunt ends so as to avoid the joining of distinct DNA fragments.

In some embodiments, the step 203 further includes the use of an apurinic/apyrimidinic endonuclease to treat damaged dsDNA prior to end repair and A-tailing in addition to a DNA ligase suitable for nick repair. One example apurinic/apyrimidinic endonuclease is APEI. The APEI enzyme catalyzes the formation of a nick in the phosphodiester backbone of dsDNA at abasic sites. When the nicks present on the backbone includes a hydroxyl group on the 5′ terminus, there may be too much steric hindrance for a polynucleotide kinase enzyme to phosphorylate this 5′ terminus, thereby preventing a DNA ligase such as Taq DNA ligase from repairing the nick. Without being limited by any particular theory, it is anticipated that treatment with APEI may be effective to remove the unphosphorylated nucleobase present at the 5′ terminus at the nick site, thereby creating a single nucleotide gap. Thereafter, T4 DNA polymerase may be used to fill in the single nucleotide gap, and the resulting product may then be repaired with Taq DNA ligase. Moreover, in the case of gaps of more than a single nucleotide, T4 DNA polymerase may be used to fill in these larger gaps, with the resulting product being similarly repaired with Taq DNA ligase.

In a step 204 of the method 200, the sample DNA is treated with one or more enzymes to convert fragmented DNA to repaired DNA having 5′ phosphorylated, 3′ dA-tailed ends. Suitable enzymes include DNA polymerases having 3′ exonuclease activity, such as a Taq DNA polymerase I derived from Thermus aquaticus, T4 DNA polymerase, T4 polynucleotide kinase, and Klenow fragment.

In some embodiments, the step 204 includes the use of a methyltransferase to restore methylation signal. In one example, the methyltransferase is DNMT1. Cytosine bases at CpG sites in human DNA are methylated on both strands (i.e., symmetric methylation). During replication, a methyltransferase such as DNMT1 can restore the specific methylation pattern on the daughter strand in accordance with that of the parental DNA by methylating hemimethylated DNA. The methylation specificity of DNMT1 can be leveraged to restore the methylation signal. In one aspect, methylation signal may be lost due to damage to the sample DNA. In another aspect, methylation signal may be lost during nick translation during the step 204. In some embodiments, DNMT1 enzyme can be added in combination with a composition for end repair and A-tailing. In some embodiments, DNMT1 enzyme can be added following the completion of end repair and A-tailing and prior to ligation of nucleic acid adapters in a step 206. Accordingly, the step 204 can include two sub-steps carried out in serial (i.e., an end repair and A-tailing sub-step followed by a methylation restoration sub-step). In some embodiments, the sample DNA product of the step 204 can be purified away from any enzymes, salts, buffers or other reagents used in the step 204. Furthermore, in some embodiments, the sample DNA product of either or both of the sub-steps of the step 204 can be purified away from any enzymes, salts, or other reagents used in the respective sub-step.

In a step 206, nucleic acid adapters may be added to enable downstream sequencing workflows. In general, addition of adapters to the repaired sample DNA from the step 204 includes an enzymatic ligation step. Accordingly, the adapters can include 5′ phosphorylated, 3′ dA-tailed ends compatible with the sample DNA; however, other approaches known in the art can also be applied. Example adapters include Y-adapters, dumbbell adapters, blunt adapters, overhang adapters and the like. Adapters may further include sample barcodes or identification sequences to distinguish one sample DNA from another, unique molecular identification sequences to distinguish between individual DNA molecules, and the like. For EM-seq, bisulfite sequencing, and other methylation detection approaches, it may be useful to provide adapters in which some or all of the cytosines present in the sample are converted or otherwise provided as 5mC. According to the method 200 of the present disclosure, it may be useful to provide adapters in which some or all of the cytosine bases present in the sample are converted from 5mC to a derivative thereof.

In one aspect, it has been shown that different methylcytosine dioxygenase enzymes exhibit sequence context bias unique to that enzyme. As a result, oxidation efficiency can vary depending on the sequence identify of the input DNA. Sequences for which methylcytosine dioxygenase exhibits a lower oxidation efficiency will therefore have less protection from deamination, for example, when treated with a cytidine deaminase. In order to mitigate the potential sequence dependency of a given methylcytosine dioxygenase enzyme, sequencing adapters may be provided in which each 5mC is converted to or otherwise replaced with 5hmC. Adapters in which each methylcytosine is 5hmC can allow for greater protection from deamination and result in greater yield (e.g., due to improved consistency in adapter sequence) and better clustering on flow cell-based sequencing instruments during. Accordingly, the step 206 can include the use of adapters in which some or all of the cytosine bases present in the sample are converted or otherwise provided as 5hmC. In other embodiments of the step 206, it may be useful to provide adapters in which some or all of the cytosine bases present in the sample are converted or otherwise provided as 5gmC for the same aforementioned reasons. In one aspect, the sample DNA product of the step 206 can be purified away from any enzymes, salts, buffers or other reagents used in the step 206.

In a step 208 of the method 200, the sample DNA including the adapters applied thereto in the step 206, is treated to convert the 5mC and 5hmC present in the DNA, and optionally, in the adapter nucleic acids, to a modified methylcytosine. In one embodiment, the step 208 is substantially similar to or the same as the step 108 of the method 100.

In a step 210, the sample DNA product of the step 208, including the converted 5mC and converted 5hmC, is treated with a cytidine deaminase in order to selectively convert unmethylated or otherwise unmodified cytosine bases present in the sample DNA to uracil. In some embodiments, the step 210 is substantially similar to or the same as the step 110 of the method 100. In some embodiments, the step 210 further includes enhancement of the deamination process through the addition of a reagent for inducing or maintaining the formation of single stranded DNA (ssDNA). In one example, reagent is a helicase, a single stranded binding protein, or the like. In one aspect, the single stranded binding protein is a T4 Gene 32 Protein. Without being limited by any particular theory, it has been observed that cytidine deaminases preferentially convert deoxycytidine in ssDNA to deoxyuridine with low activity on dsDNA. In one aspect, addition of a helicase or single stranded protein during deamination in the step 210 can ensure the substrates retain their single stranded conformation and drive the deamination reaction to completion.

In a step 212, the sample DNA resulting from the step 210 is amplified using PCR. In one embodiment, the step 212 is substantially similar to or the same as the step 112 of the method 100.

In a step 214, the sequences of the products of the PCR amplification step 212 are determined. In one embodiment, the step 214 is substantially similar to or the same as the step 114 of the method 100.

Notably, the embodiments of the method 200 according to the present disclosure can include one or more additional steps or omit one or more of the illustrated steps of the method 200. In general, the method 200 can be modified in any suitable way that still enables the outcome of providing a modified sample DNA that enables distinguishing of methylated cytosines from unmethylated cytosines. Yet other variations of the method 200 that fall within the scope of the present disclosure will be apparent from the additional examples and description included herein.

### EXAMPLES

The following Examples are meant to be illustrative and are not intended to be limiting in any way.

### Example 1

In the present example, the library preparation workflow was applied for ligation of nucleic acid adapters using either an engineered DNA ligase or a wild-type T4 DNA ligase.

In some embodiments, engineered ligase enzymes may offer improvements compared to naturally occurring (i.e., wild type) ligase enzymes in the methods described here including improved conversion efficiency (i.e., efficiency of ligation of adapter to a target in the formation of an adapter ligated target product) at very low sample input amounts, and improved ligation specificity which results in a decreased sensitivity to adapter concentrations and a reduced observation of adapter dimer. Another advantage of the use of engineered ligase enzymes is the reduction of the ligation reaction time from multiple hours (for example, overnight ligation) with a naturally occurring DNA ligase to less than 1 hour (for example, 5 minutes) with an engineered DNA ligase.

In some embodiments, the engineered DNA ligase is selected to provide enhanced ligation activity. In one aspect, the ligase is engineered for samples yielding low concentrations of DNA for input into a method according to the present disclosure. Samples that may yield low concentrations of DNA include cell-free DNA, circulating tumor DNA, DNA isolated from circulating tumor cells, circulating fetal DNA, DNA isolated from virally infected cells, fine-needle aspirates, or single cells isolated by FACS (fluorescence activated cell sorting), laser-capture microscopy, or microfluidic devices.

In some embodiments, an engineered DNA ligase is selected for use with lower concentrations of nucleic acid adapters. In one aspect, it may be useful to provide lower adapter concentrations in a ligation reaction mixture to minimize the production of adapter dimers (i.e., a first adapter ligated directly to a second adapter). Exemplary engineered DNA ligases for use according to the methods of the present disclosure include those disclosed in U.S. Patent Application Publication number 2018/0320162 to Miller et al., filed on 7 May 2018, which is herein incorporated by reference. In one aspect, an engineered DNA ligase for ligation of nucleic acid adapters as disclosed herein is distinct from a DNA ligase for nick/gap repair as in the step 203 of the method 200. For example

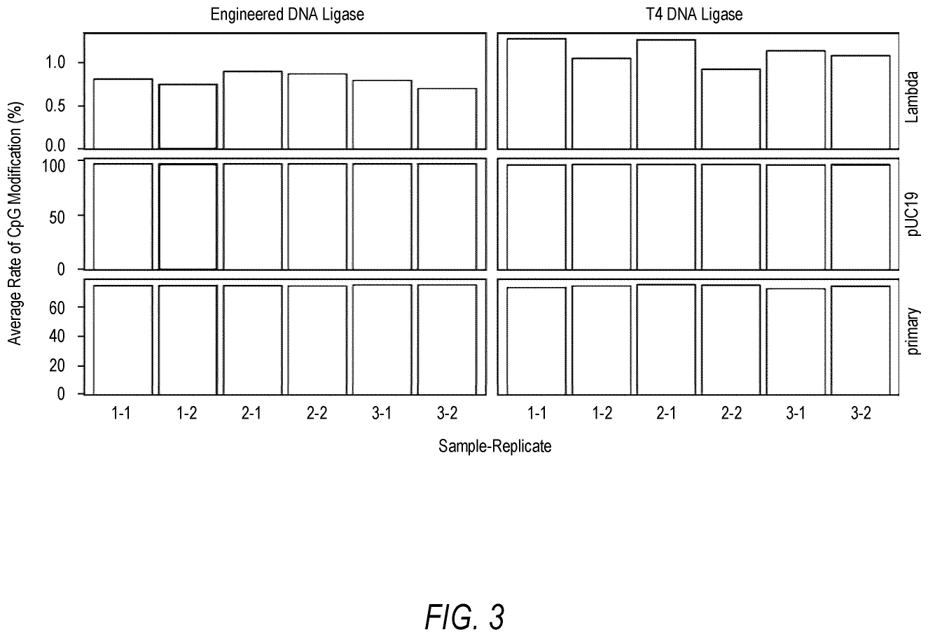

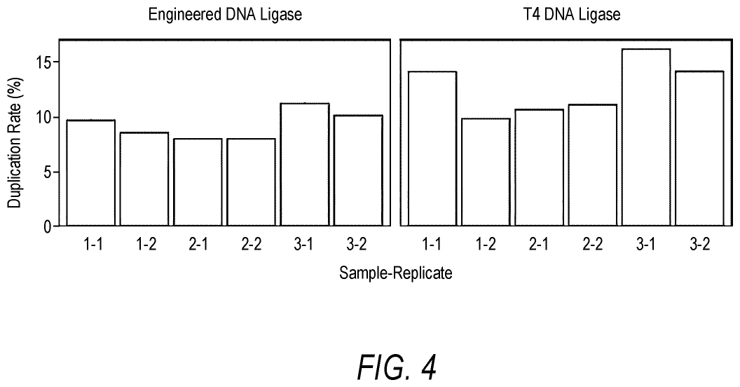

With reference to FIGS. 3 and 4, the use of an engineered DNA ligase in a methylation sequencing workflow demonstrates that not only are the results in multiple sequencing metrics comparable to a wild-type T4 DNA ligase, the engineered DNA ligase additionally improves the conversion efficiency from 98.87% to 99.19% as compared to the unmethylated lambda DNA spike-in control (conversion efficiency was calculated as follows: 100-[methylation detection percentage of unmethylated lambda DNA]). Here, a sequencing ready library was prepared from 2 ng of purified cfDNA from each of 3 healthy donors using either an engineered DNA ligase or a wild-type T4 DNA ligase in the library preparation workflow. Samples were otherwise prepared for sequencing using standard library preparation and EM-seq workflows according to the method 100. The molecule recovery of the samples prepared using the engineer DNA ligase also demonstrates an improvement to the duplication rate (i.e., the fraction of mapped reads where any two reads share the same 5′ and 3′ coordinates) compared to a wild type T4 DNA ligase.

### Example 2

The present example demonstrates the application of Taq DNA ligase and apurinic/apyrimidinic endonuclease I during the end-repair step of nucleic acid sequencing library preparation.

In general, cfDNA contains nicks (i.e., discontinuities in the dsDNA molecule where there is no phosphodiester bond between adjacent nucleotides of one strand of the dsDNA). Nicks in dsDNA, including cfDNA can provide replication initiation sites for DNA polymerase, enabling a process known as nick translation. When nick translation occurs, DNA polymerase elongates the 3′ hydroxyl terminus of the nick site, removing nucleotides by 5′ to 3′ exonuclease activity, and replacing them with dNTPs. Accordingly, the strand of the ds DNA located 3′ of the nick, including any methylated or otherwise modified bases, is absent from the product of nick translation, and the corresponding methylation signal is lost.

According to the present disclosure, modification of the method for library preparation for methyl-sequencing enables damage to the dsDNA to be repaired before nick translation can take place. In one aspect, initially excluding DNA polymerase from end repair and A-tailing reactions, and treating the sample with at least one of a DNA ligase and an apurinic/apyrimidinic endonuclease could help retain this methylation signal. After end repair, the Taq DNA polymerase is added to complete A-tailing prior to adapter ligation. In one aspect, the ligase preferably catalyzes the formation of a phosphodiester bond between the 5′-phosphate and the 3′-hydroxyl of two adjacent DNA strands in a nicked dsDNA. In particular, the ligase should preferentially catalyze bond formation when the strands to be ligated are hybridized and accurately paired, with no gap, to a complementary DNA strand. One example of a suitable ligase for nick repair is Taq DNA ligase from a Thermus species.

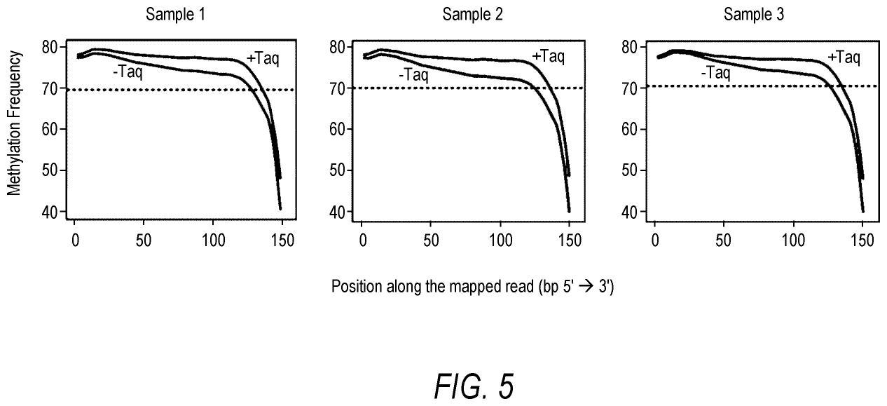

To demonstrate the application of nick repair prior to A-tailing, 2 ng of purified cfDNA from 3 healthy donors was treated with or without Taq DNA ligase derived from Thermus thermophilus HB8. In particular, end repair and A-tailing reactions were carried out in the absence of DNA polymerase, followed by treatment of the sample with Taq DNA ligase. Thereafter, Taq DNA polymerase was added to complete A-tailing prior to adapter ligation. A sequencing library was then prepared from the Taq DNA ligase treated DNA using a method according to the present disclosure. In particular, the sample DNA was further prepared for adapter ligation in an end repair and A-tailing step, followed by ligation of 5mC adapters, conversion of 5mC/5hmC, deamination of cytosine and PCR amplification as described with respect to the method 100. With reference to FIG. 5, the data show that the addition of Taq DNA ligase greatly improves the methylation signal at the 3′-end of the sequencing template and reduces methylation bias.

As used herein, the term “methylation bias” or “M-bias” refers to deviations from the expected average methylation level per position in a sequencing read. Under ideal conditions, the average methylation level should be constant across all positions within a sequencing read; however, different factors can contribute to variability in the average methylation level, thereby resulting in M-bias. In one example, methylation signal can be lost due to processing steps during collection and preparation of nucleic acids for sequencing. This loss of methylation signal (also referred to as hypomethylation) can reduce the measured average methylation level. This effect is typically observed at the ends of a sequencing read as seen in FIG. 5, wherein the methylation frequency (plotted on the y-axis), drops off significantly towards the 3′ end of the sequencing read (i.e., approximately starting at position 125 on the x-axis). For each sample in FIG. 5, the addition of Taq DNA ligase (+Taq) resulted in an increase the measured average methylation level at the 3′ end of the sequencing reads relative to the samples processed without Taq DNA ligase (−Taq). Overall, the methylation frequency for the +Taq experiments was more uniform, having a more constant average methylation level across sequencing reads, indicating an overall improvement in M-bias.

In another aspect, an apurinic/apyrimidinic endonuclease may be used to treat damaged dsDNA prior to end repair and A-tailing in addition to a ligase suitable for nick repair. One example apurinic/apyrimidinic endonuclease is APEI. The APEI enzyme catalyzes the formation of a nick in the phosphodiester backbone of dsDNA at abasic sites. When the nicks present on the backbone includes a hydroxyl group on the 5′ terminus, there may be too much steric hindrance for a polynucleotide kinase enzyme to phosphorylate this 5′ terminus, thereby preventing Taq DNA ligase from repairing the nick. Without being limited by any particular theory, it is anticipated that treatment with APEI may be effective to remove the unphosphorylated nucleobase present at the 5′ terminus at the nick site. Thereafter, T4 DNA polymerase may be used to fill in the 1 nucleotide gap, and the resulting product may then be repaired with Taq DNA ligase.

### Example 3

The present example demonstrates the use of methyltransferase to restore methylation signal.

The cytosines at CpG sites in human DNA are methylated on both strands (i.e., symmetric methylation). During replication, the methyltransferase enzyme DNMT1 restores the specific methylation pattern on the daughter strand in accordance with that of the parental DNA by methylating hemimethylated DNA. The methylation specificity of DNMT1 can be leveraged to restore the methylation signal erased by nick translation during the end-repair step of sequencing library preparation. The DNMT1 enzyme can be added following the completion of end-repair and A-tailing, and prior to ligation of sequencing adapters to restore the symmetric methylation pattern, thereby improving methylation signal detected by sequencing.

### Example 4

The present example demonstrates the use of hydroxymethylated adapters for greater protection of deamination.

It has been shown that different TET enzymes exhibit sequence context bias unique to that enzyme. As a result, oxidation efficiency can vary depending on the sequence identify of the input DNA. Sequences for which TET exhibits a lower oxidation efficiency will therefore have less protection from deamination, for example, when treated with APOBEC3A. In order to mitigate the potential sequence dependency of a given TET enzyme, sequencing adapters may be provided in which each 5mC is converted to or otherwise replaced with 5hmC. Adapters in which each methylcytosine is 5hmC would allow for greater protection from deamination and result in greater yield (e.g., due to improved consistency in adapter sequence) and better clustering on flow cell-based sequencing instruments during sequencing (e.g., Illumina-based sequencing platforms).

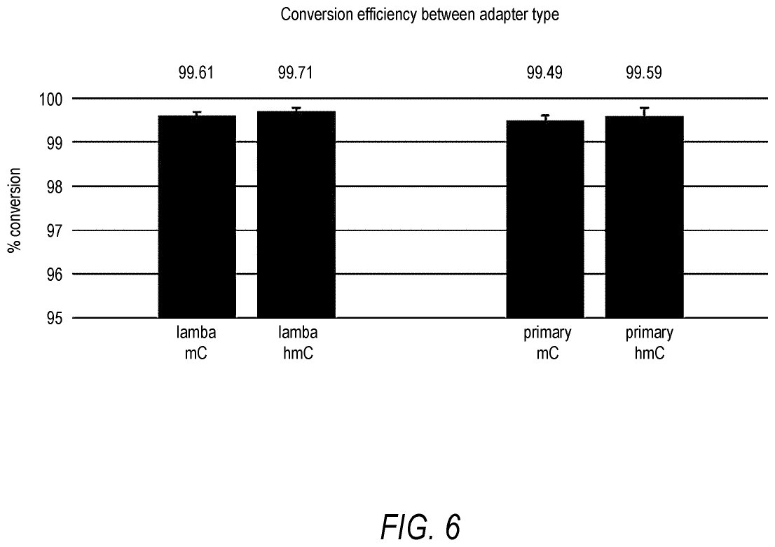

Referring to FIG. 6, the conversion efficiency between 5mC sequencing adapters (i.e., sequencing adapters in which each methylcytosine is a 5mC) and 5hmC sequencing adapters (i.e., sequencing adapters in which each methylcytosine is a 5hmC) demonstrates that using a 5hmC adapter results in higher overall conversion of methylated cytosine and reduced deamination to uracil by a cytidine deaminase in an EM-seq workflow. Conversion efficiency was determined by analyzing the conversion of cytosine to uracil with reference to unmethylated lambda genomic DNA and all non-CpG contexts in the human genome (as cytosine methylation only occurs on cytosines of CpG dinucleotides in humans). For clarity, non-deaminated cytosine would be detected as methylcytosine (i.e., a false positive).

### Example 5

The present example illustrates the use of glucosylated hydroxymethylated adapters for greater protection from deamination during EM-seq library preparation.

In one aspect, sequencing adapters for use according to the present disclosure may be further protected from deamination by converting or otherwise substituting each methylcytosine (e.g., 5mC, 5hmC) with 5gmC. For example, 5mC sequencing adapters may be treated with T4-BGT resulting in 5gmC containing adapters. This ensures that the 5hmC present in the sequencing adapters do not become further oxidized to 5fC, which may not be completely protected from deamination.

### Example 6

The present example demonstrates the addition of at least one of a helicase and an ssDNA binding protein such as T4 Gene 32 Protein during deamination to provide the substrates molecules in a single stranded format.

The apolipoprotein B mRNA editing enzyme, catalytic polypeptide-like-3 family enzymes, which include APOBEC3A, are cytidine deaminases that function as innate immune response factors through their binding and deamination of viruses and transposons, which repress the replication of these elements. Cytidine deaminase can convert deoxycytidine in ssDNA to deoxyuridine with low activity on dsDNA. Addition of a helicase, an ssDNA binding protein, or a combination thereof during deamination could help ensure the substrates retain their ssDNA conformation and drive the deamination reaction to completion.

### Example 7

The present example describes reducing the cytidine deamination reaction volume to enhance deamination efficiency.

In some embodiments it may be useful to reduce the volume of the deamination reaction to effectively increase the concentration of the reagents and drive the deamination reaction to completion. In one aspect, the total amount of the dsDNA present in the reaction may be equal to or less than about 10 nanograms (ng). In another aspect, the total amount of dsDNA present in the reaction may be equal to or less than about 1 ng, 100 picograms (pg), 10 pg, or 1 pg. In another aspect, the concentration of dsDNA present in the reaction may be equal to or less than about 1 ng, 100 pg, 10 pg, or 1 pg per 100 microliters (μL), In another aspect, the concentration of the cytidine deaminase is from about 0.05 micromolar (μM) to about 0.5 μM. In another aspect, the concentration of the cytidine deaminase is from about 0.1 μM to about 0.2 μM. In another aspect, the total volume of the composition is less than about 100 μL, 50 μL, 25, or 10 μL.

### Example 8

The present example describes the elimination of a nucleic acid purification step following cytidine deamination to improve recovery of unique library members during sample preparation for sequencing.

In one aspect, using the unpurified product of the deamination reaction as the substrate for PCR amplification may result in better recovery of unique molecules for sequencing as purification may otherwise result in loss of a portion of the sample DNA.

### Example 9

The present example demonstrates another use of methyltransferase to restore methylation signal.

As already discussed above, the cytosines at CpG sites in human DNA are methylated on both strands (i.e., symmetric methylation). During replication, DNMT1 (encoded by the DNMT1 gene in humans) restores the specific methylation pattern by methylating hemimethylated DNA. While DNMT1 is one example of a methyltransferase useful for methylating hemimethylated DNA, other methyltransferase enzymes may also be used for restoration of hemimethylated DNA, including in vitro. Another example of a suitable methyltransferase according to the present disclosure is DNMT5 from Cryptococcus neoformans. In one aspect, C. neoformans DNMT5 has been characterized as a highly specific, maintenance-type CpG methyltransferase that mediates long-term epigenome evolution. The C. neoformans DNMT5 comprises a DNMT domain and SNF2 ATPase domain, which combination of domains is, at least in part, believed to provide the enzyme with a high degree of specificity for hemimethylated DNA (relative to, for example, human DNMT1). Without being limited by any particular theory, it is believed that hemimethylated DNA preferentially stimulates ATPase activity to trigger structural reconfigurations of the methyltransferase catalytic pocket and enable cofactor binding, completion of cytosine base flipping, and catalysis. By contrast, bound unmethylated DNA does not open the catalytic pocket of the DNMT5 enzyme and is instead ejected upon ATP binding, thereby driving high fidelity (i.e., specificity for hemimethylated CpG sites in DNA as compared with unmethylated CpG sites in DNA).

In the present example, 5 ng of purified cfDNA was prepared for sequencing according to one of three distinct methods. In a first method, standard library preparation was carried out according to steps 102-112 of the method 100. More particularly, 5 ng of purified cfDNA from 2 healthy donors was subject to end repair and A-tailing reactions followed by the addition of Taq DNA polymerase to complete A-tailing prior to adapter ligation. A sequencing library was then prepared from the resulting DNA via ligation of 5mC adapters, conversion of 5mC/5hmC, deamination of cytosine and PCR amplification as described with respect to the method 100. The second method was identical to the first method with the addition of Taq DNA ligase derived from Thermus thermophilus HB8 for nick/gap repair following end repair and A-tailing, but prior to the addition of Taq DNA polymerase. Finally, the third method was identical to the second method with the addition of C. neoformans DNMT5 following the addition of Taq DNA ligase but prior to the addition of Taq DNA polymerase as in the step 204 of the method 200. Libraries prepared according to each of the three methods were then sequenced to determine the effect of C. neoformans DNMT5 on methylation bias.

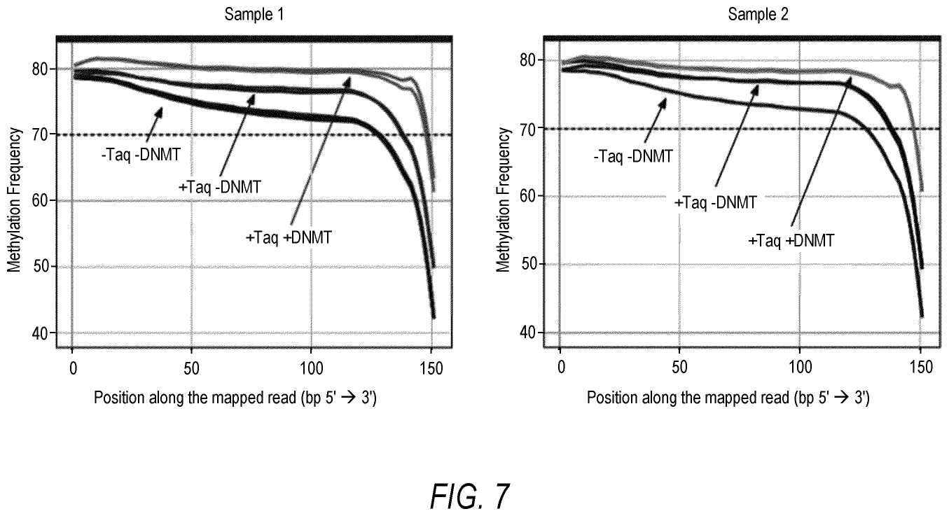

With reference to FIG. 7, the resulting data illustrated that the addition of C. neoformans DNMT5 resulted in a positive impact on M-bias even beyond that observed with the addition of Taq DNA ligase as in the experiments described in Example 2 and FIG. 5. In particular, for each sample in FIG. 5, the addition of both Taq DNA ligase and C. neoformans DNMT5 (+Taq +DNMT) resulted in an increase in the measured average methylation level at the 3′ end of the sequencing reads relative to both the samples processed without Taq DNA ligase (−Taq −DNMT) and the samples processed with Taq DNA ligase but without C. neoformans DNMT5 (+Taq −DNMT). Overall, the methylation frequency for the +Taq +DNMT5 experiments was more uniform, exhibiting a more uniform average methylation level across sequencing reads, indicating an overall improvement in M-bias.

The schematic flow charts shown in the Figures are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed in the Figures are provided to explain the logical steps of the method and are understood not to limit the scope of the method. Although various arrow types and line types may be employed, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The present invention is presented in several varying embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the system. One skilled in the relevant art will recognize, however, that the system and method may both be practiced without one or more of the specific details, or with other methods, components, materials, and so forth. In other instances, well-known structures, materials, or operations are not shown or described in detail to avoid obscuring aspects of the invention. Accordingly, the foregoing description is meant to be exemplary, and does not limit the scope of present inventive concepts.

Each reference identified in the present application is herein incorporated by reference in its entirety.

1. A method, wherein the method comprises the following steps:
(a) contacting: (i) a first enzyme whrein the first enzyme possesses double-stranded DNA (dsDNA) nick repair activity, with: (ii) a dsDNA, wherein the dsDNA comprises at least one nick, at least one cytosine, and at least one methylcytosine selected from 5-methylcytosine and 5- hydroxymethylcytosine, thereby repairing the at least one nick;
(b) converting the at least one methyl cytosine to a modified methylcytosine capable of base-pairing with a guanine; and
(c) converting the at least one cytosine to uracil.

2. The method of claim 1, wherein the modified methylcytosine is selected from 5-(β-glucosyloxymethyl)cytosine and 5-carboxycytosine (5caC).

3. The method of claim 1, wherein the at least one methylcytosine is converted, with a second enzyme, to the modified methylcytosine.

4. The method of claim 3, wherein the second enzyme is selected from a methylcytosine dioxygenase and a beta-glucosyltransferase.

5. The method of claim 1, wherein the at least one cytosine is converted, with a third enzyme, to uracil.

6. The method of claim 5, wherein the third enzyme is apolipoprotein B mRNA editing enzyme catalytic subunit 3A (APOBEC3A).

7. The method of claim 1, wherein the first enzyme is a Taq DNA ligase.

8. The method of claim 1, further comprising a step of ligating sequencing adaptors to the dsDNA.

9. The method of claim 1, further comprising a step of contacting the nick-repaired dsDNA with a fourth enzyme, wherein the fourth enzyme comprises at least one of DNA end-repair activity and A-tailing activity.

10. The method of claim 4, wherein the methylcytosine dioxygenase is TET methylcytosine dioxygenase 2, and wherein the beta-glucosyltransferase is T4-phage beta-glucosyltransferase.

11. A method, wherein the method comprises the following steps:
(a) contacting a Taq DNA ligase with a double-stranded DNA (dsDNA), wherein the dsDNA comprises at least one nick, at least one cytosine, and at least one methylcytosine, wherein the methylcytosine is selected from 5-methylcytosine and 5-hydroxymethylcytosine, thereby repairing the at least one nick;
(b) enzymatically converting, with a TET methylcytosine dioxygenase 2 (TET), the at least one methylcytosine to one of 5-hydroxymethylcytosine and 5-carboxycytosine;
(c) enzymatically converting, with a T4-phage beta-glucosyltransferase (T4-BGT) the 5-hydroxymethylcytosine to 5-(β-glucosyloxymethyl)cytosine; and
(d) enzymatically converting, with an apolipoprotein B mRNA editing enzyme catalytic subunit 3A (APOBEC3A), the at least one cytosine to uracil.

12. The method of claim 11, further comprising a step of ligating sequencing adaptors to the dsDNA.

13. The method of claim 11, further comprising contacting the nick-repaired dsDNA with an enzyme, wherein the enzyme comprises at least one of DNA end-repair activity and A-tailing activity.

14. The method of claim 1 or 11, wherein the step (a) further comprises contacting the dsDNA with an apurinic/apyrimidinic endonuclease.

15. A method, wherein the method comprises the following steps:
(a) contacting a first enzyme possessing methyltransferase activity with a double-stranded DNA (dsDNA), wherein the dsDNA comprises at least one hemimethylated CpG site, at least one additional cytosine, and at least one methylcytosine, wherein the methylcytosine is selected from 5-methylcytosine and 5-hydroxymethylcytosine, thereby methylating the hemimethylated CpG site, wherein the at least one additional cytosine remains unmethylated;
(b) converting the at least one methylcytosine to a modified methylcytosine capable of base-pairing with a guanine; and
(c) converting the at least one additional cytosine to uracil.

16. The method of claim 15, wherein the modified methylcytosine is selected from 5-(β-glucosyloxymethyl)cytosine and 5-carboxycytosine (5caC).

17. The method of claim 15, wherein the at least one methylcytosine is converted, with a second enzyme, to the modified methylcytosine.

18. The method of claim 17, wherein the second enzyme is selected from a methylcytosine dioxygenase and a beta-glucosyltransferase.

19. The method of claim 15, wherein the at least one additional cytosine is converted, with a third enzyme, to uracil.

20. The method of claim 19, wherein the third enzyme is apolipoprotein B mRNA editing enzyme catalytic subunit 3A (APOBEC3A).

21. The method of claim 15, wherein the first enzyme is selected from Human DNA (cytosine-5) methyltransferase (DNMT1) and Cryptococcus neoformans DNA methyltransferase (DNMT5).

22. The method of claim 15, further comprising a step of ligating sequencing adaptors to the dsDNA.

23. The method of claim 15, further comprising a step of contacting the dsDNA with a fourth enzyme, wherein the fourth enzyme comprises at least one of DNA end-repair activity and A-tailing activity.

24. The method of claim 18, wherein the methylcytosine dioxygenase is TET methylcytosine dioxygenase 2, and wherein the beta-glucosyltransferase is T4-phage beta-glucosyltransferase.

25. A method, wherein the method comprises the following steps:
(a) ligating an adapter to a double-stranded DNA (dsDNA), wherein the dsDNA comprises at least one cytosine and at least one methylcytosine, wherein the methylcytosine is selected from 5-methylcytosine and 5-hydroxymethylcytosine, wherein the adapter comprisingcomprises a nucleic acid sequencingsequence comprising at least one methylcytosine, wherein each methylcytosine present in the adapter is a 5-hydroxymethylcytosine;
(b) converting the at least one methylcytosine present in the dsDNA, and the at least one methylcytosine present in the adapter, to a modified methylcytosine capable of base-pairing with a guanine; and
(c) converting the at least one additional cytosine to uracil.

26. The method of claim 25, wherein the modified methylcytosine is selected from 5-(β-glucosyloxymethyl)cytosine and 5-carboxycytosine (5caC).

27. The method of claim 25, wherein the at least one methylcytosine is converted, with a first enzyme, to the modified methylcytosine.

28. The method of claim 27, wherein the first enzyme is selected from a methylcytosine dioxygenase and a beta-glucosyltransferase.

29. The method of claim 25, wherein the at least one cytosine is converted, with a second enzyme, to uracil.

30. The method of claim 29, wherein the second enzyme is apolipoprotein B mRNA editing enzyme catalytic subunit 3A (APOBEC3A).

31. The method of claim 25, further comprising a step of contacting dsDNA with a third enzyme, wherein the third enzyme comprises at least one of DNA end-repair activity and A-tailing activity.

32. The method of claim 28, wherein the methylcytosine dioxygenase is TET methylcytosine dioxygenase 2, and wherein the beta-glucosyltransferase is T4-phage beta-glucosyltransferase.

33. A method, wherein the method comprises the following steps:
(a) ligating an adapter to a double-stranded DNA (dsDNA), wherein the dsDNA comprises at least one cytosine and at least one methylcytosine selected from 5-methylcytosine and 5-hydroxymethylcytosine, the adapter comprising a nucleic acid sequence comprising at least one modified methylcytosine, wherein each modified methylcytosine present in the adaptor is a 5-(β-glucosyloxymethyl)cytosine;
(b) converting the at least one methylcytosine present in the dsDNA to a modified methylcytosine capable of base-pairing with a guanine; and
(c) converting the at least one additional cytosine to uracil.

34. The method of claim 33, wherein the modified methylcytosine present in the dsDNA is selected from 5-(β-glucosyloxymethyl)cytosine and 5-carboxycytosine (5caC).

35. The method of claim 33, wherein the at least one methylcytosine in the dsDNA is converted, with a first enzyme, to the modified methylcytosine.

36. The method of claim 35, wherein the first enzyme is selected from a methylcytosine dioxygenase and a beta-glucosyltransferase.

37. The method of claim 33, wherein the at least one cytosine is converted, with a second enzyme, to uracil.

38. The method of claim 37, wherein the second enzyme is apolipoprotein B mRNA editing enzyme catalytic subunit 3A (APOBEC3A).

39. The method of claim 33, further comprising a step of contacting the dsDNA with a third enzyme, wherein the third enzyme comprises at least one DNA end-repair activity and A-tailing activity.

40. The method of claim 36, wherein the methylcytosine dioxygenase is TET methylcytosine dioxygenase 2, and wherein the beta-glucosyltransferase is T4-phage beta-glucosyltransferase.

41. An adaptor for methyl-sequencing, wherein the adapter comprises a first nucleic acid having a 3′ end complementary to a 5′ end of a second nucleic acid, thereby forming a complementary region, wherein the complementary region comprises at least one modified methylcytosine, wherein each modified methylcytosine present in the adapter is a 5-(β-glucosyloxymethyl)cytosine.

42. An adaptor for methyl-sequencing, wherein the adapter comprises a first nucleic acid having a 3′ end complementary to a 5′ end of a second nucleic acid, thereby forming a complementary region, wherein the complementary region comprises at least one modified methylcytosine, wherein each modified methylcytosine present in the adapter is a 5-hyhdroxymethylcytosine.

43. A method, wherein the method comprises the following steps:
(a) combining: (i) a double-stranded DNA (dsDNA) comprising, wherein the dsDNA comprises at least one cytosine, and at least one methylcytosine selected from 5-methylcytosine and 5-hydroxymethylcytosine, (ii) a cytidine deaminase, and (iii) at least one of a single-strand DNA-binding protein and a helicase;
(b) converting the at least one methylcytosine to a modified methylcytosine capable of base-pairing with a guanine; and
(c) converting, with the cytidine deaminase, the at least one cytosine to uracil.

44. The method of claim 43, wherein the modified methylcytosine is selected from 5-(β-glucosyloxymethyl)cytosine and 5-carboxycytosine (5caC).

45. The method of claim 43, wherein the at least one methylcytosine is converted to the modified methylcytosine, with at least one of a methylcytosine dioxygenase and a beta-glucosyltransferase.

46. The method of claim 43, wherein the cytidine deaminase is apolipoprotein B mRNA editing enzyme catalytic subunit 3A (APOBEC3A).

47. The method of claim 43, further comprising a step of ligating sequencing adaptors to the dsDNA.

48. The method of claim 43, further comprising a step of contacting the dsDNA, with an enzyme having at least one of DNA end-repair activity and A-tailing activity.

49. The method of claim 45, wherein the methylcytosine dioxygenase is TET methylcytosine dioxygenase 2, and wherein the beta-glucosyltransferase is T4-phage beta-glucosyltransferase.

50. A method, wherein the method comprises the following steps:
(a) combining: (i) a double-stranded DNA (dsDNA), wherein the dsDNA comprises at least one cytosine, and at least one methylcytosine selected from 5-methylcytosine and 5-hydroxymethylcytosine, (ii) a cytidine deaminase, and (iii) at least one of a single-strand DNA-binding protein and a helicase
(b) enzymatically converting, with a TET methylcytosine dioxygenase 2 (TET), the at least one methylcytosine to one of 5-hydroxmethylcytosine and 5-carboxycytosine;
(c) enzymatically converting, with a T4-phage beta-glucosyltransferase (T4-BGT) 5-hydroxymethylcytosine, to 5-(β-glucosyloxymethyl)cytosine; and
(d) enzymatically converting, with an apolipoprotein B mRNA editing enzyme catalytic subunit 3A (APOBEC3A), the at least one cytosine to uracil.

51. The method of claim 50, further comprising a step of ligating sequencing adaptors to the dsDNA.

52. The method of claim 50, further comprising a step of contacting the dsDNA with an enzyme having at least one of DNA end-repair activity and A-tailing activity.

53. A composition, wherein the composition comprises the following:
(a) a double-stranded DNA (dsDNA) comprising at least one cytosine, and at least one modified cytosine selected from 5-carboxycytosine and 5-(β-glucosyloxymethyl)cytosine;
(b) a buffer; and
(c) a cytidine deaminase,
wherein the amount of the dsDNA is from about 1 pg to about 10 ng,
wherein the concentration of the cytidine deaminase is from about 0.05 μM to about 0.5 μM, and
wherein the total volume of the composition is less than about 100 μL.

54. A method, wherein the method comprises the following steps:
(a) providing a first composition, wherein the first composition comprises a double-stranded DNA (dsDNA) comprising at least one cytosine, and at least one methylcytosine, wherein the methylcytosine is selected from 5-methylcytosine and 5-hydroxymethylcytosine;
(b) enzymatically converting, with a TET methylcytosine dioxygenase 2 (TET), the at least one methylcytosine to one of 5-hydroxymethylcytosine and 5-carboxycytosine, thereby forming a first dsDNA product;
(c) enzymatically converting, with a T4-phage beta-glucosyltransferase (T4-BGT) 5-hydroxymethylcytosine, in the first dsDNA product to 5-(β-glucosyloxymethyl)cytosine, thereby forming a second dsDNA product;
(d) enzymatically converting, with an apolipoprotein B mRNA editing enzyme catalytic subunit 3A (APOBEC3A), the at least one cytosine in the second dsDNA product to uracil, thereby forming a methyl-sequencing product composition comprising the APOBEC3A and a third dsDNA product;
(e) combining the methyl-sequencing product composition with an amplification composition; and
(f) amplifying, by polymerase chain reaction, the third dsDNA product, wherein the amplifying occurs in the presence of the APOBEC3A.

55. A method according to any one of claims 1, 11, 15, 25, 33, 43, 50, and 54, further comprising a step of ligating an adapter to the dsDNA, with an engineered DNA ligase.

56. (canceled)

57. A method, wherein the method comprises the following steps:
(a) combining:
a first enzyme wherein the first enzyme possesses nick repair activity,
a second enzyme wherein the second enzyme possesses 5′ to 3′ polymerase activity, the second enzyme lacking 5′ to 3′ exonuclease activity,
a third enzyme possessing, wherein the third enzyme possesses apurinic/apyrimidinic endonuclease activity,
a double-stranded DNA (dsDNA) wherein the dsDNA comprises at least one single-stranded discontinuity selected from a nick and a gap, at least one cytosine, and at least one methylcytosine selected from 5-methylcytosine and 5-hydroxymethylcytosine, and
a plurality of deoxynucleoside triphosphates (dNTPs);
(b) repairing the at least one discontinuity in the dsDNA;
(c) converting the at least one methylcytosine to a modified methylcytosine capable of base-pairing with a guanine; and
(d) converting the at least one cytosine to uracil.
